(19) United States
(12) Patent Application Publication
(10) Pub. No.: US 2021/0177992 A1
(43) Pub. Date: Jun. 17, 2021

(54) ANTICANCER FLUORESCENT SUBSTANCE DERIVED FROM NATURAL MATERIALS

(71) Applicant: SEOUL NATIONAL UNIVERSITY R&DB FOUNDATION, Seoul (KR)

(72) Inventors: Seong Keun KIM, Gwanak-gu, Seoul (KR); Hyunsook LEE, Seocho-gu, Seoul (KR); Jong Woo LEE, Songpa-gu, Seoul (KR); Sangjin PAIK, Seocho-gu, Seoul (KR); Ilseung YANG, Sejong (KR); Doyk HWANG, Jungnang-gu, Seoul (KR); Inai PARK, Seocho-gu, Seoul (KR); Jiho PARK, Gwanak-gu, Seoul (KR); Jung Eun LEE, Michuhol-gu, Incheon (KR); Eunhak LIM, Yangcheon-gu, Seoul (KR); Seon Jin HWANG, Hwaseong-si, Gyeonggi-do (KR)

(21) Appl. No.: 16/771,061

(22) PCT Filed: Dec. 6, 2018
(86) PCT No.: PCT/KR2018/015453

§ 371 Date: Jun. 9, 2020

(30) Foreign Application Priority Data
Dec. 11, 2017 (KR) 10-2017-0168978

(51) Int. Cl. A61K 49/00; A61K 31/704; A61K 31/12; G01N 33/50

(52) U.S. Cl. CPC A61K 49/0021; A61K 49/0002; G01N 33/5011; A61K 31/12; A61K 31/704

(57) ABSTRACT

Provided is a composition for preventing or treating cancer, the composition including resveratrone, resveratrone glucoside, or a combination thereof as an active ingredient.

### TECHNICAL FIELD

The present disclosure relates to a pharmaceutical composition for preventing or treating cancer and a contrast agent composition for diagnosing cancer, each composition including resveratrone or resveratrone glucoside as an active ingredient.

### BACKGROUND ART

MRI, CT, and X-ray techniques, which are existing non-fluorescent technologies used for cancer surgery, are disadvantageous in that they not only produce radioactive waste but also are difficult to use for real-time observation during surgery, and due to low resolution during cancer surgery, result in excessive resection of normal tissues or a low removal rate of cancer. There are reports that the probability of remaining cancer cells after surgery is practically more than 40% in breast cancer and more than 80% in pancreatic cancer [Nat Rev Clin Oncol, 10, 507-518 (2013)]. Because of these disadvantages, the use of fluorescence-guided cancer surgery techniques is becoming increasingly popular due to their high resolution and signal sensitivity [Nature Medicine, 17, 1315-1319 (2011)]. Biocompatibility of the fluorescent substance used in this case is an important factor in the surgical techniques. Existing quantum dots are well known for toxicity due to their size and heavy metals, and most organic fluorescent substances used are not free from cytotoxicity [Nature Methods, 5, 763-775 (2008), Mol Imaging, 8, 341-54 (2009)]. Due to such biocompatibility problems, there is an increasing demand for fluorescent substances having higher compatibility.

The research on fluorescent substances reported to date is only for combining technical compositions, each separately synthesized: a technical composition that plays a role in treating cancer, such as an anticancer agent, production of reactive oxygen species through photoreaction, and the photothermal effect, and a technical composition having a diagnostic role in measuring signals such as fluorescence, etc. Such a synthesis/combination method requires several preparation steps, and thus it is necessary to reduce the cost and to simplify the preparation method for the development of a hybrid functional fluorescent substance having anticancer properties. In response to the demand, the present inventors have developed a natural product-based fluorescent substance having anticancer properties.

“Resveratrone” represented by the following [Formula 1] is a compound derived from reveratrol which is known to be abundant in peanuts, grapes, berries, etc.

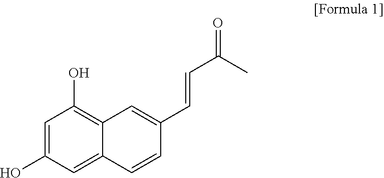 [SMILES: CC(=O)/C=C/c1ccc2cc(O)cc(O)c2c1]

Further, “resveratrone glucoside” represented by the following [Formula 2] is a fluorescent compound in which a glucose group is bound to resveratrone.

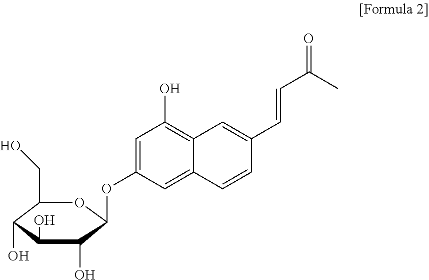

Korean Patent No. 10-1294993 (Yang et al, Photochemical generation of a new, highly fluorescent compound from non-fluorescent resveratrol, Chem Commun, 2012, 48, 3839-3841) and Korean Patent No. 10-1244176 (Yang et al, Live bio-imaging with fully biocompatible organic fluorophores, Journal of Photochemistry & Photobiology, B: Biology 166 (2017) 52-57) describe a method of preparing resveratrone and resveratrone glucoside, but do not disclose diagnostic use thereof.

Korean Patent Publication No. 10-2008-0104927 and Korean Patent No. 10-1074026 disclose a drug delivery system, in which a fluorescent substance such as iron oxide or doxorubicin is bound to an anticancer agent, but do not disclose use of a compound having both diagnostic and therapeutic effects, because an additional process of polymer preparation is needed.

Given this background, the present inventors have made intensive efforts to develop a compound including resveratrone or resveratrone glucoside and capable of simultaneously diagnosing cancer and preventing and treating cancer, thereby completing the present disclosure.

### PRIOR ART DOCUMENTS

### Patent Documents

(Patent Document 0001) Korean Patent No. 10-1294993 (registered in 20130805)
(Patent Document 0002) Korean Patent No. 10-1244176 (registered in 20100311)
(Patent Document 0003) Korean Patent Publication No. 10-2008-0104927 (published in 20081203)
(Patent Document 0004) Korean Patent No. 10-1074026 (registered in 20111010)

### Non-Patent Documents

(Non-Patent Document 0001) Yang et al, Photochemical generation of a new, highly fluorescent compound from non-fluorescent resveratrol, Chem Commun, 2012, 48, 3839-3841.
(Non-Patent Document 0002) Yang et al, Live bio-imaging with fully biocompatible organic fluorophores, Journal of Photochemistry & Photobiology, B: Biology 166 (2017) 52-57.
(Non-Patent Document 0003) Nat Rev Clin Oncol, 10, 507-518 (2013)
(Non-Patent Document 0004) Nature Medicine, 17, 1315-1319 (2011)
(Non-Patent Document 0005) Nature Methods, 5, 763-775 (2008), Mol Imaging, 8, 341-54 (2009)

### DESCRIPTION OF EMBODIMENTS

### Technical Problem

An aspect provides a composition for preventing or treating cancer, the composition including resveratrone, resveratrone glucoside, or a combination thereof as an active ingredient.

Another aspect provides a method of preventing or treating cancer, the method including administering resveratrone, resveratrone glucoside, or a combination thereof to a subject in need of treatment.

Still another aspect provides a contrast agent composition including resveratrone, resveratrone glucoside, or a combination thereof as an active ingredient.

Still another aspect provides a method of detecting the presence of cancer cells, the method including administering resveratrone, resveratrone glucoside, or a combination thereof to a subject; and measuring fluorescence intensity in the subject.

Still another aspect provides a method of screening for a cancer therapeutic drug, the method including contacting resveratrone, resveratrone glucoside, or a combination thereof with cancer cells; culturing the cancer cells; and measuring fluorescence intensity in the cancer cell culture.

### Solution to Problem

Hereinafter, the present disclosure will be described in more detail.

Unless defined otherwise, all technical terms used herein have the same meanings as those generally understood by one of ordinary skill in the art to which the present disclosure belongs. Further, although methods or samples are described herein, those similar or equivalent thereto are also incorporated in the scope of the present disclosure. The numerical values described herein are considered to include the meaning of “about”, unless otherwise specified. The contents of all the publications disclosed as references herein are incorporated in the present disclosure.

An aspect provides a composition for preventing or treating cancer, the composition including resveratrone, resveratrone glucoside, or a combination thereof as an active ingredient.

In a specific embodiment, the composition may be used as a contrast agent.

The term “contrast agent” refers to a drug that is administered to the human body so that a particular tissue or blood vessel is easily visualized during a diagnostic imaging examination or procedure. In the following Examples, it was demonstrated that resveratrone or resveratrone glucoside has low toxicity to normal cells while having toxicity to cancer cells (Examples 1 and 2). It was also demonstrated that since resveratrone or resveratrone glucoside may emit fluorescence, it may be used in cell detection, and in particular, it may specifically detect cancer cells (Example 3). Therefore, the composition according to a specific embodiment may be used for treating or preventing cancer, and at the same time, it may be used as a contrast agent. The composition according to a specific embodiment does not adversely affect normal cells in detecting the presence or absence of cancer cells in vivo as well as in vivo, and thus it is possible to safely and significantly diagnose cancer cells. In addition, since the composition is toxic to cancer cells, it may simultaneously achieve preventive and therapeutic effects.

In a specific embodiment, the composition may be simultaneously used for detecting cancer. The detecting of cancer may be to provide information for diagnosing cancer, or to diagnose cancer or to monitor prognosis of cancer.

When the composition is used for cancer detection or as a contrast agent, fluorescence excitation and emission wavelength ranges of the composition may be as in FIG. 1B. The fluorescence excitation wavelength range may be about 200 nm to about 480 nm, and the fluorescence emission wavelength range may be about 450 nm to about 700 nm. The fluorescence excitation and emission wavelength ranges may vary depending on a subject to be measured, and may be adjusted depending on a substance included in the composition, other than resveratrone or resveratrone glucoside. Fluorescence of resveratrone and resveratrone glucoside may be measured by single photon absorption as well as two photon absorption (FIG. 8). Therefore, when the composition is used for cancer detection or as a contrast agent, fluorescence emitted from the composition may be measured by single photon microscopy and/or two photon microscopy. When the two photon microscopy is used, the composition may be particularly useful for observation of cells in living tissue. When the two photon absorption phenomenon is used, a pulse laser of 600 nm to 900 nm, 700 nm to 850 nm, 750 nm to 850 nm, or about 800 nm may be used.

Resveratrone or resveratrone glucoside may be used independently or selectively as an active ingredient, and may also be used in a combination thereof. The composition may further include one or more of active ingredients exhibiting the same or similar functions. The composition may be administered in various oral and parenteral dosage forms during practical clinical administration. When formulated, a commonly used diluent or excipient such as a filler, an extender, a binder, a wetting agent, a disintegrating agent, a surfactant, etc. may be used in the preparation. Solid formulations for oral administration include tablets, pills, powders, granules, capsules, etc., and the solid formulations are prepared by mixing with at least one excipient, for example, starch, calcium carbonate, gelatin, etc. Liquid formulations for oral administration include suspensions, liquids for internal use, emulsions, syrups, etc. In addition to simple diluents commonly used, such as water and liquid paraffin, various excipients, for example, a wetting agent, a sweetener, a flavoring agent, a preservative, etc., may be included. Formulations for parenteral administration include sterile aqueous solutions, non-aqueous solvents, suspensions, emulsions, lyophilized preparations, and suppositories. For non-aqueous solvents and suspensions, propylene glycol, polyethylene glycol, a vegetable oil such as olive oil, and an injectable ester such as ethyl oleate may be used. As bases for suppositories, Witepsol, macrogol, Tween 61, cacao butter, laurin butter, glycerogelatin, etc. may be used. Meanwhile, the pharmaceutical composition according to the present disclosure may be formulated with an appropriate carrier depending on the route of administration. The carrier includes all kinds of solvents, dispersion media, oil-in-water or water-in-oil emulsions, aqueous compositions, liposomes, microbeads, and microsomes. Examples of the appropriate carriers may include sugars, including lactose, dextrose, sucrose, sorbitol, mannitol, xylitol, erythritol, maltitol, etc., starches, including corn starch, wheat starch, rice starch, potato starch, etc., celluloses including cellulose, methyl cellulose, sodium carboxymethyl cellulose, hydroxypropylmethyl cellulose, etc., fillers such as gelatin, polyvinyl pyrrolidone, etc. In addition, crosslinked polyvinylpyrrolidone, agar, alginic acid, sodium alginate, etc. may optionally be added as a disintegrating agent. Furthermore, the pharmaceutical composition may further include an anticoagulant, a lubricant, a wetting agent, a flavoring agent, an emulsifier, a preservative, etc. In addition, the pharmaceutical composition according to the present disclosure may further include one or more buffers (e.g., saline or PBS), carbohydrates (e.g., glucose, mannose, sucrose or dextran), antioxidants, bacteriostatic agents, chelating agents (e.g., EDTA or glutathione), adjuvants (e.g., aluminum hydroxide), suspending agents, thickening agents, diluents, and/or preservatives.

The composition may include a carrier and a vehicle for a contrast agent, which are commonly used in the medical field. Specifically, the carrier and the vehicle may include ion exchange resins, alumina, aluminum stearate, lecithin, serum proteins (e.g., human serum albumin), buffer substances (e.g., various phosphates, glycine, sorbic acid, potassium sorbate, partial glyceride mixtures of saturated vegetable fatty acids), water, salts, or electrolytes (e.g., protamine sulfate, disodium hydrogen phosphate, potassium hydrogen phosphate, sodium chloride, zinc salts), colloidal silica, magnesium trisilicate, polyvinylpyrrolidone, cellulose-based substrates, polyethylene glycol, sodium carboxymethylcellulose, polyarylate, waxes, polyethylene glycol, wool fat, etc., but are not limited thereto. The composition may further include a lubricant, a wetting agent, an emulsifier, a suspending agent, an excipient, a diluent, a preservative, etc., in addition to the above components. In a specific embodiment, the composition may further include radioisotopes, quantum dots, MRI contrast agents, or diagnostic antibodies, but is not limited thereto.

The composition may be prepared as an aqueous solution for parenteral administration, and buffer solutions such as Hanks' solution, Ringer's solution, or physically buffered saline may be used. Aqueous injection suspensions may be added with a substrate capable of increasing viscosity of the suspension, such as sodium carboxymethyl cellulose, sorbitol, or dextran. Further, the composition may be in the form of a sterile injectable preparation of a sterile injectable aqueous or oily suspension. Such a suspension may be formulated using an appropriate dispersing or wetting agent (e.g., Tween 80) and a suspending agent according to techniques known in the art. The sterile injectable preparation may also be a sterile injectable solution or suspension in a nontoxic parenterally acceptable diluent or solvent (e.g., a solution in 1,3-butanediol). Applicable vehicles and solvents include mannitol, water, Ringer's solution, and an isotonic sodium chloride solution. In addition, sterile nonvolatile oils are usually used as a solvent or a suspending medium. For this purpose, any of low irritant nonvolatile oils including synthetic mono- or diglycerides may be used. When the composition according to a specific embodiment is used as a contrast agent, it may be administered to a living body or a sample, and the image may be obtained by detecting auto-fluorescent signals emitted from the living body or the sample.

The administration dosage of the composition varies depending on the patient's body weight, age, sex, health status, diet, administration time, administration mode, excretion rate, disease severity, etc. In addition, the composition may be administered in combination with a known compound having the effect of preventing or treating a cancer disease.

In a specific embodiment, the composition may include resveratrone glucoside, or may include a combination of resveratrone glucoside and resveratrone.

In the following examples, it was confirmed that when both of resveratrone and resveratrone glucoside are used, both of them exhibit fluorescence, thereby being used in the detection of cancer cells. In particular, it was confirmed that resveratrone glucoside emits remarkably strong fluorescence in MCF7 cells, as compared with normal cells (Example 3-1 and Example 3-3). Glucose transporters that transport glucose into cells exist on the cell membrane. In general, since cancer cells have higher cell activities than normal cells, glucose transporters are highly activated, and the intracellular transport of glucose through these transporters is significantly higher than that of normal cells. Since resveratrone glucoside is able to pass through the glucose transporters of cancer cells, cancer cells tend to have a significantly higher level of intracellular resveratrone glucoside than normal cells. According to this property, cancer cells may be distinguished from normal cells by fluorescence. Thus, for specific detection of cancer cells, resveratrone glucoside may be included. Meanwhile, when resveratrone and resveratrone glucoside are included in combination, it may be more appropriate to detect the presence of cancer cells and to achieve the effect of preventing or treating cancer at the same time, because resveratrone is more toxic to cancer cells than resveratrone glucoside (see Table 1, etc.).

In a specific embodiment, the cancer may be selected from the group consisting of solid cancer, primary cancer, metastatic cancer, and recurrent cancer.

The term “cancer”, which is a cellular proliferative disease, collectively refers to a neoplastic disease caused by a tumor. Specifically, the cancer has a glucose transporter activation level of 2 times or more, 3 times or more, 5 times or more, 10 times or more, 20 times or more, 50 times or more, or 100 times or more than that of normal cells. The glucose transporter activation level may be measured using a known technique, and for example, may be determined by a method of measuring and comparing cancer cell-specific fluorescence intensity. The cancer disease includes, but is not limited to, solid cancer, primary cancer, metastatic cancer, and recurrent cancer, such as colon cancer, spleen cancer, rectal cancer, lung cancer, liver cancer, stomach cancer, esophageal cancer, pancreatic cancer, gallbladder cancer, kidney cancer, bladder cancer, prostate cancer, testicular cancer, cervical cancer, endometrial cancer, choriocarcinoma, ovarian cancer, breast cancer, thyroid cancer, brain cancer, head and neck cancer, malignant melanoma, lymphoma, bone marrow cancer, soft tissue sarcoma, spinal cancer, but is not limited thereto.

Another aspect provides a method of preventing or treating cancer, the method including administering resveratrone, resveratrone glucoside, or a combination thereof to a subject in need of treatment.

In a specific embodiment, the method of preventing or treating cancer may further include measuring fluorescence intensity in the subject.

The measuring of fluorescence intensity may be performed by X-ray fluorescence analysis, an electron probe micro analyzer, scanning electron microscopy, auger electron microscopy, flow cytometry, single photon microscopy, or two photon microscopy, but is not limited thereto. Fluorescence excitation and emission ranges for measuring the fluorescence intensity are the same as described above.

In a specific embodiment, the method of preventing or treating cancer may also detect or diagnose cancer.

Among the terms or elements mentioned in the method of preventing or treating cancer, the same as those mentioned in the description of the composition for preventing or treating cancer claimed are understood to be identical.

Still another aspect provides a contrast agent composition including resveratrone, resveratrone glucoside, or a combination thereof as an active ingredient.

In a specific embodiment, the contrast agent composition may be for specifically detecting cancer.

Among the terms or elements mentioned in the contrast agent composition, the same as those mentioned in the description of the composition for preventing or treating cancer claimed are understood to be identical.

Still another aspect provides a method of detecting the presence of cancer cells, the method including administering resveratrone, resveratrone glucoside, or a combination thereof to a subject; and measuring fluorescence intensity in the subject.

In a specific embodiment, the method may be to diagnose cancer in the subject.

In a specific embodiment, the method may be to provide information for diagnosing cancer in the subject.

The method may be performed in vitro or in vivo. When the method according to a specific embodiment is performed in vitro, the composition may be administered to a tissue or cell isolated from the subject, or a cancer cell isolated from the culture thereof. When the method according to a specific embodiment is performed in vivo, the composition may be orally or parenterally administered to a living subject.

In a specific embodiment, the method may be for detecting cancer and to prevent or treat cancer at the same time.

In a specific embodiment, the subject may be a mammal or a human.

In a specific embodiment, the subject may be cells cultured in vitro, tissue isolated from a body, an organoid, or a combination thereof.

Among the terms or elements mentioned in the method of detecting cancer cells, the same as those mentioned in the description of the composition for preventing or treating cancer and the contrast agent composition claimed are understood to be identical.

Still another aspect provides a method of screening for a cancer therapeutic drug, the method including contacting resveratrone, resveratrone glucoside, or a combination thereof with cancer cells; culturing the cancer cells; and measuring fluorescence intensity in the cancer cell culture.

Since the composition according to a specific embodiment is non-toxic to normal cells, but toxic to cancer cells, and the compound itself emits fluorescence within a specific range, it may be used in screening for a drug having excellent cancer therapeutic efficiency and low toxicity to normal cells in an easy, economical, and significant manner.

Among the terms or elements mentioned in the method of screening for the drug, the same as those mentioned in the description of the composition for preventing or treating cancer or the method of detecting cancer cells claimed are understood to be identical.

### Advantageous Effects of Disclosure

A composition for preventing or treating cancer according to an aspect, the composition including resveratrone, resveratrone glucoside, or a combination thereof as an active ingredient may be used to safely prevent or treat cancer without toxicity to normal cells. Since the composition for preventing or treating cancer according to a specific embodiment may be used as a contrast agent or may specifically detect cancer, it may be used to treat the cancer while safely monitoring prognosis of the cancer.

A contrast agent composition including resveratrone, resveratrone glucoside, or a combination thereof as an active ingredient according to another aspect may be used as a contrast agent or may be used for specifically detecting cancer without adverse effects on normal cells, thereby diagnosing cancer.

According to a method of detecting the presence of cancer cells using the composition according to still another aspect, the presence of cancer cells may be detected in a subject in vivo or in vitro, and thus the method may be safely employed in diagnosing cancer in the subject or in vitro study. The composition according to a specific embodiment has no toxicity to normal cells, thereby providing high significance for diagnosing or detecting cancer.

According to a method of screening for a cancer therapeutic agent using the composition according to still another aspect, cancer therapeutic agent candidates may be efficiently and safely identified.

### BRIEF DESCRIPTION OF DRAWINGS

FIG. 1A is an illustration showing anticancer effect and cancer cell detection ability of resveratrone and resveratrone glucoside, and FIG. 1B shows excitation and emission wavelength ranges of resveratrone and resveratrone glucoside;

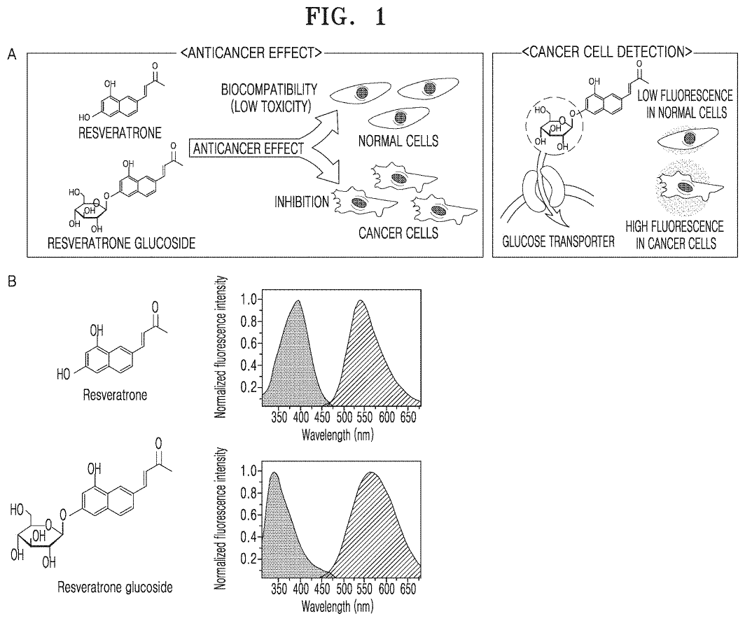

FIG. 2A shows fractions of cells showing apoptosis after treatment of normal cells and cancer cells with resveratrone, and FIG. 2B shows FACS graphs showing fractions of cells showing apoptosis after treatment with resveratrone glucoside, in which the right section of each graph represents an apoptosis zone;

FIG. 3A and FIG. 3B show graphs of TUNEL analysis results of observing apoptotic signals after treatment with resveratrone and resveratrone glucoside, respectively;

FIG. 4 shows graphs of Western blotting results of examining apoptosis after treatment of normal breast cells and breast cancer cells with resveratrone;

FIG. 5 shows optical microscope images (DIC) showing cell morphology after treatment of normal breast cells and breast cancer cells with resveratrone;

FIG. 6A shows fluorescence microscope images showing fluorescence intensity after treatment of normal breast cells and breast cancer cells with resveratrone glucoside; FIG. 6B shows a graph of measuring and comparing fluorescence intensity after treatment of normal breast cells and breast cancer cells with resveratrone glucoside;

FIG. 7 shows a fluorescence microscope image showing HeLa cells treated with resveratrone;

FIG. 8A and FIG. 8B show optical microscope (DIC) and two photon fluorescence microscope images which were observed over time after treatment of Zebrafish embryos with resveratrone and resveratrone glucoside, respectively; and

FIG. 9 shows optical microscope (DIC) and fluorescence microscope images of observing apoptotic effects after treatment of organoids with resveratrone.

### MODE OF DISCLOSURE

Hereinafter, the present disclosure will be described in detail with reference to the examples. However, the following examples are only for illustrating the present disclosure, and the content of the present disclosure is not limited by the following examples.

### Example 1: Measurement of Cancer Cell Apoptosis by Resveratrone and Resveratrone Glucoside

1-1. Measurement by Fluorescence-Activated Cell Sorting (FACS)

Cancer cell apoptotic effects of resveratrone and resveratrone glucoside (see U.S. Pat. No. 9,708,23762) were measured by FACS using Annexin-V binding to phosphatidiylserine (PS) which exists in the inner leaflet of the cytoplasmic membrane and is exposed to the outer leaflet of cell membrane during apoptosis.

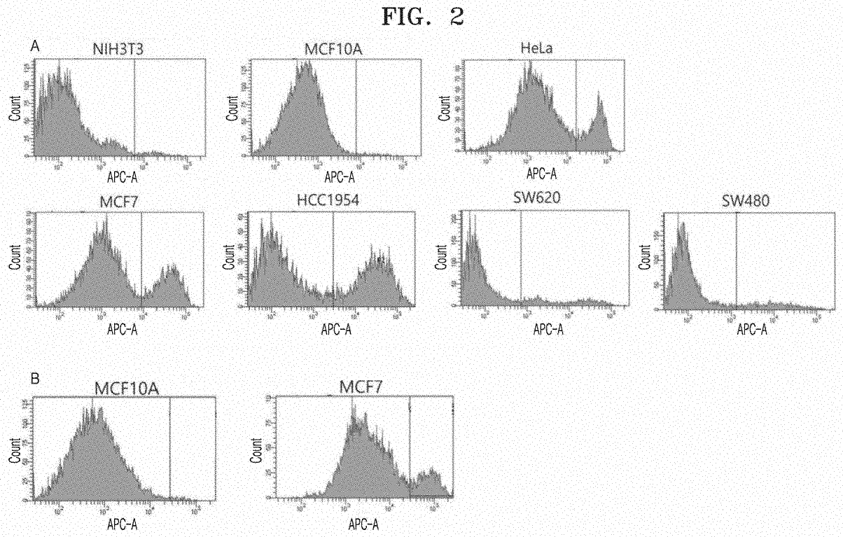

In detail, apoptosis of normal cells or cancer cells, each treated with resveratrone or resveratrone glucoside, was evaluated by measuring signals of Alexa dye-conjugated annexin V using FACS. HeLa, MCF7, SW480, SW620, and HCC1954 which are cancer cells, and NIH3T3 and MCF10A which are normal cells, each treated with 50 μM resveratrone or 100 μM resveratrone glucoside, were cultured under conditions of 37° C. and 5% CO₂ for 24 hours. Thereafter, media was removed, and the cells were washed with PBS buffer twice to remove remaining resveratrone or resveratrone glucoside. The cells were collected and centrifuged to remove supernatants. Annexin-bound buffer (10 mM HEPES, 140 mM NaCl, 2.5 mM CaCl₂, pH 7.4) was added and resuspended. 5 μl of Alexa Fluor 647-conjugated annexin V was added to the resuspended cells at the cell volume of 100 μl, followed by incubation for 15 minutes. 1 ml of binding buffer was added to the incubated cells, followed by centrifugation. The supernatant was removed to remove remaining Alexa Fluor 647-conjugated annexin V. Thereafter, 500 μl of annexin-binding buffer was further added, and cells were passed through a cell strainer cap and stored in a round-bottom tube. The number of apoptotic cells in each 1000 cells of normal cells and cancer cells was counted by repeated FACS analysis using an excitation wavelength of 633 nm, at which Alexa Fluor 647 emits fluorescence, but resveratrone does not emit fluorescence (FIGS. 2A and 2B).

As a result, it was confirmed that, as compared with that of normal cells, the number of apoptotic cells in the cancer cells was increased to at least 15 times to about 40 times when treated with resveratrone, and increased to about 22 times when treated with resveratrone glucoside (Table 1).

TABLE 1

| | resveratrone | | | | | | | resveratrone glucoside | |
|---|---|---|---|---|---|---|---|---|---|
| | Kind of cell | | | | | | | | |
| | | | | | | | | normal | cancer |
| | normal cell | | cancer cell | | | | | cell | cell |
| | NIH3T3 | MCF10A | HeLa | MCF7 | SW480 | SW620 | HCC1954 | MCF10A | MCF7 |
| Apoptotic | 2.0 | 1.0 | 20.0 | 26.0 | 15.5 | 17.4 | 39.3 | 0.7 | 15.5 |
| fraction | | | | | | | | | |
| (%) | | | | | | | | | |

1-2. Measurement by TUNEL Assay Cancer cell apoptotic effects of resveratrone and resveratrone glucoside, which were evaluated in Example 1-1, were re-evaluated by TUNEL assay.

In detail, a cancer cell MCF7 and a normal cell MCF10A were treated with 50 μM of resveratrone or resveratrone glucoside, respectively, and incubated for 24 hours. Thereafter, the cells were treated with 0.5% PBS-Triton X-100 and incubated for 10 minutes to increase cell permeability. The cells were treated with terminal deoxynucleotide transferase and tetra-methyl-rhodamine dUTP, followed by incubation at 37° C. for 1 hour. Next, the cells were washed with PBS three times, and treated with DAPI dye for nuclear counterstaining. Apoptotic signals were observed using fluorescence images.

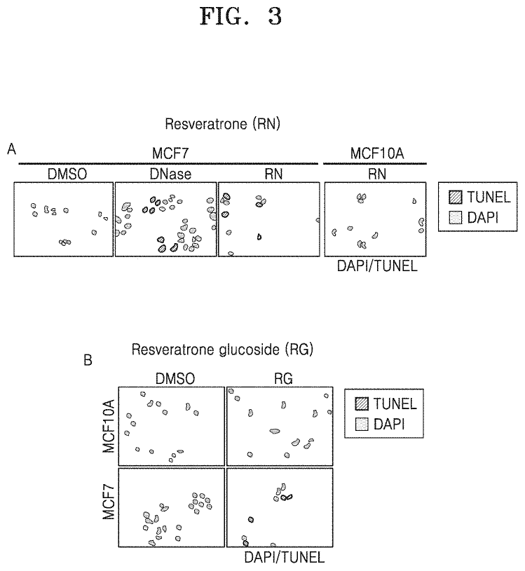

As a result, TUNEL detecting apoptotic signals was observed only in the cancer cell MCF7, indicating that resveratrone or resveratrone glucoside had no effect on normal cells and exhibited selective apoptotic ability against cancer cells (FIGS. 3A and 3B).

### Example 2: Measurement of Cancer Cell Apoptosis Induction of Resveratrone

2-1. Measurement by Western Blotting

Cancer cell apoptotic effects of resveratrone and resveratrone glucoside, which were evaluated in Examples 1-1 and 1-2, were re-evaluated by Western blotting.

In detail, a cancer cell MCF7 and a normal cell MCF10A were treated with 50 μM of resveratrone, respectively, and incubated for 48 hours. The incubated cells were harvested and lysed with a NETN buffer (150 mM NaCl, 20 mM Tris/Cl pH 8.0, 0.5% v/v NP-40, 1 mM EDTA) containing a protease inhibitor, and then conjugated with a cleaved parp antibody, and sequentially, with a secondary antibody. Then, the presence or absence thereof was examined.

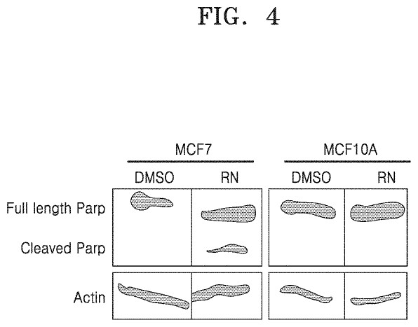

As a result, Cleaved parp which was not observed in the resveratrone-treated normal cells was observed in the resveratrone-treated cancer cells, indicating that resveratrone induced apoptosis of cancer cells (FIG. 4).

2-2. Measurement by Optical Microscopy

Cancer cell apoptotic effects of resveratrone and resveratrone glucoside, which were evaluated in the above Examples, were examined by observing morphology.

In detail, a normal cell MCF10A and a cancer cell MCF7 were treated with 50 μM of resveratrone, respectively, and incubated for 48 hours. After incubation, media was removed, and the cells were washed with PBS buffer three times. Changes of cell morphology in PBS buffer were observed and compared by optical microscopy.

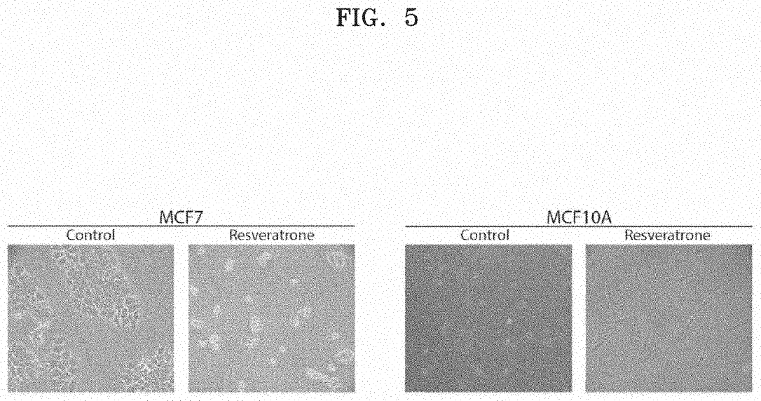

As a result, cell morphology of the normal cell MCF10A was clearly observed irrespective of treatment with resveratrone, whereas the resveratrone-treated cancer cell MCF7 showed morphological features of apoptosis, as compared with the control group (FIG. 5).

### Example 3. Evaluation of Function of Resveratrone and Resveratrone Glucoside as Contrast Agent

3-1. Resveratrone Glucoside

Function of resveratrone glucoside as a contrast agent was evaluated.

In detail, a cancer cell MCF7 and a normal cell MCF10A were treated with 300 μM of resveratrone glucoside, respectively, and incubated for 3 hours. Then, they were washed with PBS buffer twice, and cells were resuspended in PBS buffer, respectively. Imaging of each cell suspension was performed by confocal fluorescence microscopy using 800 nm pulse laser as a light source and a two-photon absorption phenomenon of resveratrone, and mean fluorescence intensity of resveratrone glucoside in the normal cell and cancer cell was measured and compared.

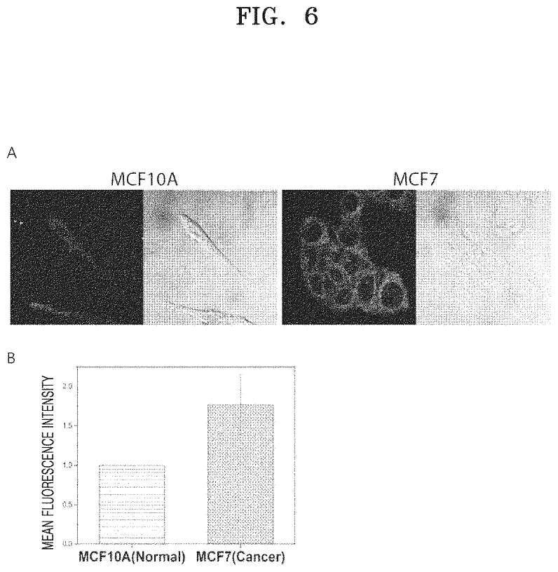

As a result of the experiment, the cancer cell MCF7 showed stronger fluorescence intensity than the normal cell MCF10A (FIG. 6A), and the intensity was about 170% stronger (FIG. 6B). Therefore, it is suggested that resveratrone glucoside may be used to diagnose cancer cells by distinguishing cancer cells from normal cells.

3-2. Resveratrone

Function of resveratrone as a contrast agent was evaluated.

In detail, media was removed from HeLa cell culture. The cells were washed with PBS buffer three times, and treated with MeOH at 20° C., and fixed by incubation for 5 minutes. Thereafter, the cells were treated with 30 uM of resveratrone and incubated for 4 hours. Then, the cells were washed with PBS buffer twice and treated with Mowiol which is an imaging medium. The Mowiol-treated HeLa cells were excited at 405 nm, and a fluorescence image of resveratrone was examined.

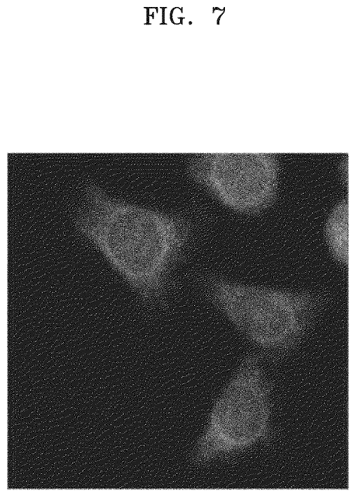

As a result, it was confirmed that resveratrone may also function as a contrast agent (FIG. 7).

3-3. Evaluation of Cytotoxicity of Resveratrone as Contrast Agent

It was examined whether resveratrone exhibited cytotoxicity when used as a contrast agent.

In detail, zebrafish embryos (donated by Hyunsook Lee Lab, School of Biological Sciences, Seoul National University) were cultured, and treated with 300 μM of resveratrone or resveratrone glucoside. At 2 hr, 20 hr, 48 hr, and 72 hr after treatment, their changes were observed.

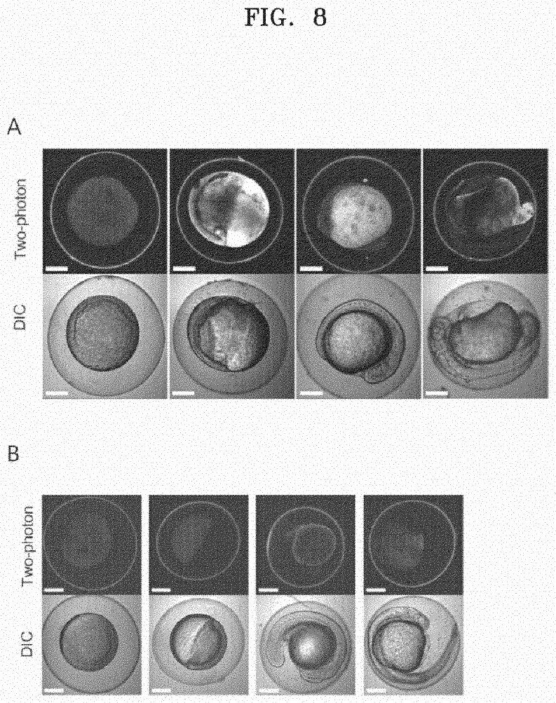

As a result, resveratrone induced no cytotoxicity during development of zebrafish embryos, indicating no toxicity as a contrast agent (FIG. 8A). Resveratrone glucoside also induced no cytotoxicity during development of zebrafish embryos, but weak fluorescence was observed, as compared with resveratrone (FIG. 8B).

### Example 4. Examination of Resveratrone Effect in Organoids

As an alternative to animal experiments, it was confirmed that resveratrone had a significant cancer cell apoptotic effect on organoids.

In detail, as an alternative to a pancreatic tissue of a wild-type mouse and an individual with cancer, a pancreatic tissue having G12D mutation of Kras gene was obtained from a transgenic mouse, and the pancreatic tissue was dissociated with a dissociation solution, filtered. Then, a cell pellet containing ductal cells was collected by centrifugation, and incubated with a biological substrate material. The dissociation solution containing Hank's Balanced Salt Solution (HBSS) (3 ml), collagenase P (5 mg/ml), and DNase 1 (1 Units/μl) was used in a volume of 3 ml per pancreatic tissue (Vpan=about 1.08 mg/mm³). When the collected cell pellet was incubated with the biological substrate material, three-dimensional pancreatic organoids were prepared by incubation under conditions of 37° C. and 5% CO₂ with Matrigel which is a gelatinous protein mixture secreted from the murine Engelbreth-Holm-Swarm (EHS) sarcoma. Resveratrone and resveratrone glucoside were treated using DMSO as a solvent to reach a specific concentration with respect to the culture. After 72 hr, the medium was replaced by a fresh culture medium, and the above treatment was repeated. 12 hr after final treatment, changes of the organoids were recorded using an inverted fluorescence microscope (available from Zeiss) capable of photographing 450 mm fluorescence. To examine morphology of the organoids, differential interference contrast (DIC) images were also photographed.

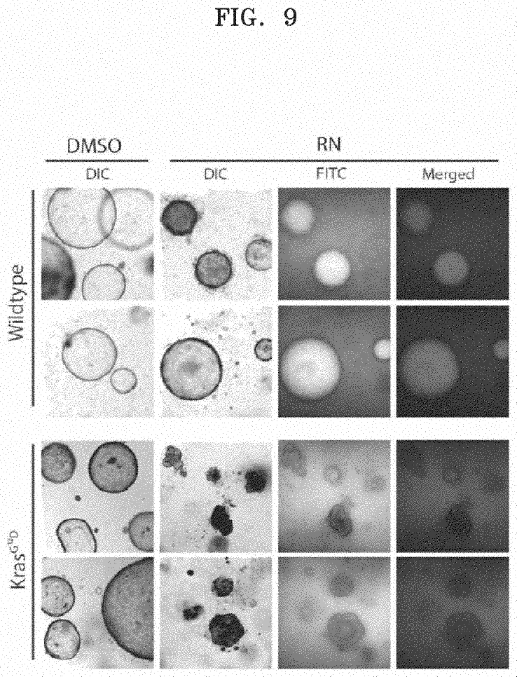

As a result, unlike mouse pancreatic organoids, organoids having G12D mutation of Kras gene showed apoptosis induction in response to resveratrone (FIG. 9). Accordingly, it is predicted that the effects identical or similar to those demonstrated in the present Example may also be obtained in vivo.

As described above, the present disclosure has been described with reference to the examples. It will be understood by those skilled in the art to which the present disclosure pertains that the present disclosure may be embodied in modified forms without departing from essential characteristics thereof. Therefore, the disclosed embodiments are to be considered in all respects only as illustrative and not restrictive. The scope of the present disclosure is indicated by the appended claims rather than by the foregoing description. It shall be interpreted that all differences within the equivalent scope are included in the present disclosure.

1-13. (canceled)

14. A method for preventing or treating cancer, the method comprising administering resveratrone, resveratrone glucoside, or a combination thereof to a subject in need thereof.

15. A method of claim 14, wherein the cancer is selected from the group consisting of solid cancer, primary cancer, metastatic cancer, and recurrent cancer.

16. A method of claim 14, wherein the method further includes measuring fluorescence intensity in the subject.

17. A method of claim 14, wherein the method is for detecting or diagnosing cancer.

18. A pharmaceutical composition for preventing or treating cancer, the composition comprising resveratrone, resveratrone glucoside, or a combination thereof as an active ingredient.

19. The composition of claim 18, wherein the composition further comprises radioisotopes, quantum dots, MM contrast agents, or diagnostic antibodies.

20. The composition of claim 18, wherein the composition further comprises a pharmaceutically acceptable carrier, excipient, or diluent.

21. A contrast agent composition comprising resveratrone, resveratrone glucoside, or a combination thereof as an active ingredient.

22. The contrast agent composition of claim 21, wherein the composition is for specifically detecting cancer.

23. The contrast agent composition of claim 21, wherein the composition further includes radioisotopes, quantum dots, MM contrast agents, or diagnostic antibodies.

24. The contrast agent composition of claim 21, wherein the composition is administered to a living body or a sample.

25. A method for detecting the presence of cancer cells, the method comprising:
administering resveratrone, resveratrone glucoside, or a combination thereof to a subject; and
measuring fluorescence intensity in the subject.

26. The method of claim 25, wherein the subject is a mammal or a human.

27. The method of claim 25, wherein the subject is cells cultured in vitro, tissue isolated from a body, an organoid, or a combination thereof.

28. A method for screening for a cancer therapeutic drug, the method comprising:
contacting resveratrone, resveratrone glucoside, or a combination thereof with cancer cells;
culturing the cancer cells; and
measuring fluorescence intensity in the cancer cell culture.
